(19) United States
(12) Patent Application Publication
(10) Pub. No.: US 2024/0091562 A1
(43) Pub. Date: Mar. 21, 2024

(54) ACOUSTIC SHOCK WAVE OR PRESSURE PULSE TREATMENT AND METHODS OF USE FOR BRAIN INFLAMMATION

(71) Applicant: SoftWave Tissue Regeneration Technologies, Inc., Kennesaw, GA (US)

(72) Inventor: John F. Warlick, Kennesaw, GA (US)

(21) Appl. No.: 17/949,725

(22) Filed: Sep. 21, 2022

(51) Int. Cl. A61N 1/36; A61H 23/00

(52) U.S. Cl. CPC A61N 1/36025; A61H 23/008; A61H 23/0245

(57) ABSTRACT

A method of treating a traumatic brain injury to reduce pressure and inflammation using pressure pulses or shock waves having the steps of placing an applicator head of an acoustic shock wave or pressure pulse generator or source on a head near a swollen region at the brain injury; coupling the applicator head directly or indirectly to an exposed surface of the skin and head near the swollen region; and activating the generator or source to emit pressure pulses or acoustic shock waves through the skin and head to brain tissue exhibiting high pressure and inflammation to reduce the pressure and inflammation.

### FIELD OF THE INVENTION

The present invention relates to a treatment for delivering acoustic shock waves or pressure pulses to brain tissue non-invasively and methods used in conjunction with the device to treat the brain for swelling and inflammation.

### BACKGROUND OF THE INVENTION

Brain swelling, brain edema, cerebral edema, and elevated intracranial pressure refer to an increase in pressure inside the skull. The swelling can cause excess pressure or bleeding resulting in more damage.

Swelling from a brain injury is often a result of trauma to the head such as a fall or accident. Aside from a traumatic brain injury, some reasons brain swelling may occur include blood clot or stroke, brain hemorrhage, infections, tumors, etc.

For symptoms of brain swelling, especially after a traumatic brain injury, a neurologist using diagnosis techniques, from physical head, neck and neurological exams to CT and MRI scans will be able to determine the extent of swelling and provide a course of treatment.

Minor brain swelling can resolve on its own as long as the brain continues to receive adequate supplies of blood and oxygen. If pressure is limiting or preventing adequate supply, medical intervention may be necessary. Some of the common treatments for brain swelling include oxygen therapy which helps ensure that the blood has enough oxygen in it, which can help control swelling; some medications can help treat brain swelling by decreasing the likelihood of clot formation; intravenous fluids help prevent blood pressure from dropping too low; ventriculostomy which is an operative procedure that involves cutting a small hole in the skull to drain cerebrospinal fluid to help relieve pressure; decompressive craniectomy which helps to relieve pressure by removing a portion of the skull; or surgically addressing the site of the problem by removing a tumor or repairing a damaged artery which is causing the pressure.

In practice, the transmission of acoustic shock waves and pressure pulses works extremely well in fluids. The wave patterns can propagate quickly in fluids when not obstructed by solid objects or voids. If a solid object is in the path of the wave pattern, the wave energy is effectively blocked to a large extent with a small fraction passing through the object. Alternatively, if the wave pattern propagates into a void or air gap, the energy is dissipated.

U.S. Pat. No. 7,507,213 B2, issued Mar. 24, 2009, entitled “Pressure Pulse/Shock Wave Therapy Method For Organs”; disclosed invasive procedures to treat organs such as the heart and the brain by surgically exposing the organ and invasively treating the organ with acoustic shock waves or pressure pulses after the surgical procedure to at least partially expose the organ or to provide a surgical access portal to the organ. The idea was to provide an unobstructed path to the tissue of the surgically exposed organ.

The same group of inventors in U.S. Pat. No. 7,544,171 B2, issued Jun. 9, 2009, entitled “Methods For Promoting Nerve Regeneration And Neuronal Growth And Elongation”; proposed a variety of diseases associated with the brain could be treated non-invasively. That patent suggested and taught that transmission of the shock waves or pressure pulses could effectively pass through the hard skull bone to treat the underlying brain tissue. The inventors were confident that the beneficial therapy could be useful for a variety of brain disorders. These inventors, while believing the benefits of such treatments were potentially great, had provided two less than ideal solutions. The first requiring invasive surgery provided a superior access. The second being non-invasive was simpler, but highly unpredictable as to what amount, if any, of the wave transmissions were getting through the bone to the brain. Since the bone thickness and hardness of the skull varies greatly, the energy passing through it is highly unpredictable. Overcoming this uncertainty by increasing the energy levels to higher levels increases the risk of brain trauma caused by the treatment.

In the present invention, a non-invasive low energy shock wave treatment is disclosed overcoming these issues.

### SUMMARY OF THE INVENTION

The device of the present invention allows for a method of treating a traumatic brain injury to reduce pressure and inflammation using pressure pulses or shock waves having the steps of placing an applicator head of an acoustic shock wave or pressure pulse generator or source on a head near a swollen region at the brain injury; coupling the applicator head directly or indirectly to an exposed surface of the skin and head near the swollen region; and activating the generator or source to emit pressure pulses or acoustic shock waves through the skin and head to brain tissue exhibiting high pressure and inflammation to reduce the pressure and inflammation.

Preferably, the emitted pressure pulses or acoustic shock waves are transmitted in a pattern passing through the head to the brain. The method allows the emitted pressure pulses or acoustic shock waves pattern to impinge the brain through the boney structure of the cranium or skull. The method has the pressure pulse being an acoustic pulse which includes several cycles of positive and negative pressure. The pressure pulse has an amplitude of the positive part of such a cycle should be above 0.1 MPa and the time duration of the pressure pulse is from below a microsecond to about a second. The rise times of the positive part of the first pressure cycle in the range of nanoseconds (ns) up to some milliseconds (ms). The pressure pulse can be the acoustic shock waves of very fast pressure pulses having amplitudes above 0.1 MPa and rise times of the amplitude being below 1000 ns. Typically, the duration of the shock wave is typically below 1-3 microseconds (μs) for the positive part of a cycle and typically above some microseconds for the negative part of a cycle.

One treatment method features subjecting the brain to convergent, divergent, planar or near planar acoustic shock waves or pressure pulses in the absence of a focal point impinging the neuronal cells stimulating a cellular response in the absence of creating cavitation bubbles evidenced by not experiencing the sensation of hemorrhaging caused by the emitted waves or pulses in neuronal cells wherein the neuronal cells are positioned within an unobstructed path of the emitted shock waves or pressure pulses; and away from any localized geometric focal volume or point of the emitted shock waves wherein the emitted shock waves or pressure pulses either have no geometric focal volume or point or have a focal volume or point ahead of the neuronal cells or beyond the neuronal cells thereby passing the emitted waves or pulses through the neuronal cells while avoiding having any localized focal point within the neuronal cells of the brain.

Ideally, the emitted pressure pulses or shock waves are convergent, divergent, planar or near planar and the pressure pulse shock wave generator or source is based on electro-hydraulic, electromagnetic, piezoceramic or ballistic wave generation having an energy density value ranging as low as 0.00001 mJ/mm² to a high end of below 1.0 mJ/mm².

The method allows for subjecting the brain directly to the acoustic shock waves with a low energy density of less than 1.0 mJ/mm² per shock wave to stimulate said neuronal cells or brain tissue wherein the neuronal cells or brain tissue is positioned directly within a path of the emitted pressure pulses or acoustic shock waves in the absence of any focal point or if a focal point exists, the neuronal cells or brain tissue being treated is positioned away from any focal point.

The method allows the energy density to be selected to avoid any cell damage to the neuronal cells or brain tissue. The method beneficially treats the brain to stimulate by accelerating or increasing neuronal cell growth or regeneration wherein the administering is applied to a patient who has a pathological condition of the brain exhibiting damage caused by injury or disease such as diabetes, brain damage associated with stroke, and for the treatment of neurological disorders related to neurodegeneration, including Parkinson's disease, Alzheimer's disease and amyotrophic lateral sclerosis, multiple sclerosis and disseminated sclerosis, any one of which has caused an increased pressure and inflammation which is reduced by the treatment. The method of treating the brain stimulates the brain by accelerating and increasing neuronal cell neurological brain tissue growth or regeneration or repair in addition to reducing brain tissue swelling and pressure and inflammation and wherein the neuronal cell or neurological brain tissue is from a mammal which is a human or an animal.

### Definitions

“Adrenergic receptor”, the adrenergic receptors or adrenoceptors are a class of G protein-coupled receptors that are targets of many catecholamines like norepinephrine (noradrenaline) and epinephrine (adrenaline) produced by the body, but also many medications like beta blockers, β2 agonists and α2 agonists, which are used to treat high blood pressure and asthma for example. Many cells have these receptors, and the binding of a catecholamine to the receptor will generally stimulate the sympathetic nervous system (SNS). SNS is responsible for the fight-or-flight response, which is triggered for example by exercise or fear causing situations. This response dilates pupils, increases heart rate, mobilizes energy, and diverts blood flow from non-essential organs to skeletal muscle. These effects together tend to increase physical performance momentarily.

Brain-derived neurotrophic factor, also known as BDNF, is a protein that, in humans, is encoded by the BDNF Gene. BDNF is a member of the neurotrophin family of growth factors, which are related to the canonical nerve growth factor. Neurotrophic factors are found in the brain and the periphery.

A “curved emitter” is an emitter having a curved reflecting (or focusing) or emitting surface and includes, but is not limited to, emitters having ellipsoidal, parabolic, quasi parabolic (general paraboloid) or spherical reflector/reflecting or emitting elements. Curved emitters having a curved reflecting or focusing element generally produce waves having focused wave fronts, while curved emitters having a curved emitting surfaces generally produce wave having divergent wave fronts.

“Divergent waves” in the context of the present invention are all waves which are not focused and are not plane or nearly plane. Divergent waves also include waves which only seem to have a focus or source from which the waves are transmitted. The wave fronts of divergent waves have divergent characteristics. Divergent waves can be created in many different ways, for example: A focused wave will become divergent once it has passed through the focal point. Spherical waves are also included in this definition of divergent waves and have wave fronts with divergent characteristics.

“Eosinophils”, sometimes called eosinophiles or, less commonly, acidophils, are a variety of white blood cells and one of the immune system components responsible for combating multicellular parasites and certain infections in vertebrates. Along with mast cells and basophils, they also control mechanisms associated with allergy and asthma. They are granulocytes that develop during hematopoiesis in the bone marrow before migrating into blood, after which they are terminally differentiated and do not multiply.

“extracorporeal” means occurring or based outside the living body.

A “generalized paraboloid” according to the present invention is also a three-dimensional bowl. In two dimensions (in Cartesian coordinates, x and y) the formula yn=2px [with n being #2, but being greater than about 1.2 and smaller than 2, or greater than 2 but smaller than about 2.8]. In a generalized paraboloid, the characteristics of the wave fronts created by electrodes located within the generalized paraboloid may be corrected by the selection of (p (−z,+z)), with z being a measure for the burn down of an electrode, and n, so that phenomena including, but not limited to, burn down of the tip of an electrode (−z,+z) and/or disturbances caused by diffraction at the aperture of the paraboloid are compensated for.

A “paraboloid” according to the present invention is a three-dimensional reflecting bowl. In two dimensions (in Cartesian coordinates, x and y) the formula y2=2px, wherein p/2 is the distance of the focal point of the paraboloid from its apex, defines the paraboloid. Rotation of the two-dimensional figure defined by this formula around its longitudinal axis generates a de facto paraboloid.

“Plane waves” are sometimes also called flat or even waves. Their wave fronts have plane characteristics (also called even or parallel characteristics). The amplitude in a wave front is constant and the “curvature” is flat (that is why these waves are sometimes called flat waves). Plane waves do not have a focus to which their fronts move (focused) or from which the fronts are emitted (divergent). “Nearly plane waves” also do not have a focus to which their fronts move (focused) or from which the fronts are emitted (divergent). The amplitude of their wave fronts (having “nearly plane” characteristics) is approximating the constancy of plain waves. “Nearly plane” waves can be emitted by generators having pressure pulse/shock wave generating elements with flat emitters or curved emitters. Curved emitters may comprise a generalized paraboloid that allows waves having nearly plane characteristics to be emitted.

A “pressure pulse” according to the present invention is an acoustic pulse which includes several cycles of positive and negative pressure. The amplitude of the positive part of such a cycle should be above about 0.1 MPa and its time duration is from below a microsecond to about a second. Rise times of the positive part of the first pressure cycle may be in the range of nanoseconds (ns) up to some milliseconds (ms). Very fast pressure pulses are called shock waves. Shock waves used in medical applications do have amplitudes above 0.1 MPa and rise times of the amplitude can be below 1000 ns, preferably at or below 100 ns. The duration of a shock wave is typically below 1-3 microseconds (μs) for the positive part of a cycle and typically above some microseconds for the negative part of a cycle.

“Shock Wave”: As used herein is defined by Camilo Perez, Hong Chen, and Thomas J. Matula; Center for Industrial and Medical Ultrasound, Applied Physics Laboratory, University of Washington, 1013 NE 40th Street, Seattle, Washington 98105; Maria Karzova and Vera A. Khokhlovab; Department of Acoustics, Faculty of Physics, Moscow State University, Moscow 119991, Russia; (Received 9 Oct. 2012; revised 16 Apr. 2013; accepted 1 May 2013) in their publication, “Acoustic field characterization of the Duolith: Measurements and modeling of a clinical shock wave therapy device”; incorporated by reference herein in its entirety.

Waves/wave fronts described as being “focused” or “having focusing characteristics” means in the context of the present invention that the respective waves or wave fronts are traveling and increase their amplitude in direction of the focal point. Per definition the energy of the wave will be at a maximum in the focal point or, if there is a focal shift in this point, the energy is at a maximum near the geometrical focal point. Both the maximum energy and the maximal pressure amplitude may be used to define the focal point.

### BRIEF DESCRIPTION OF THE DRAWINGS

The invention will be described by way of example and with reference to the accompanying drawings in which:

FIG. 1 is a simplified depiction of a pressure pulse/shock wave (PP/SW) generator with focusing wave characteristics.

FIG. 2 is a simplified depiction of a pressure pulse/shock wave generator with plane wave characteristics.

FIG. 3 is a simplified depiction of a pressure pulse/shock wave generator with divergent wave characteristics.

FIG. 4 is a simplified depiction of a pressure pulse/shock wave generator connected to a control/power supply unit.

FIG. 5 is a graph showing an exemplary ultrasound wave pattern.

FIG. 6 is a graph of an exemplary acoustic shock wave pattern.

FIG. 7 shows a patient being treated extracorporeally with shock waves being transmitted through the skin and cranial bone tissue to the region to be treated.

### DETAILED DESCRIPTION OF THE INVENTION

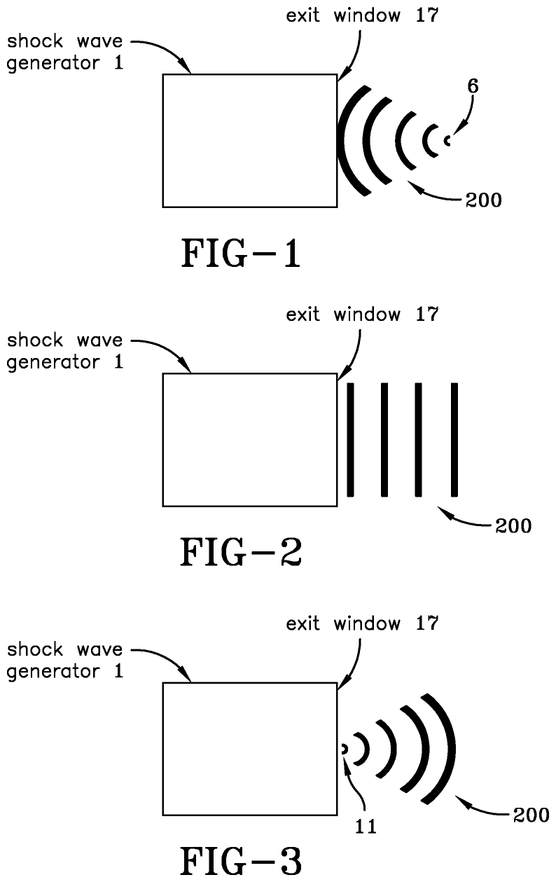

With reference to FIGS. 1-3, a variety of schematic views of acoustic shock waves or pressure pulses are described. The following description of the proper amplitude and pressure pulse intensities of the shock waves are provided along with a description of how the shock waves actually function. For the purpose of describing, the shock waves were used as exemplary and are intended to include all of the wave patterns discussed in the figures as possible treatment patterns.

FIG. 1 is a simplified depiction of a pressure pulse/shock wave (PP/SW) generator, such as a shock wave head, showing focusing characteristics of transmitted acoustic pressure pulses. Numeral 1 indicates the position of a generalized pressure pulse generator, which generates the pressure pulse and, via a focusing element, focuses it outside the housing to treat diseases. The affected tissue or organ is generally located in or near the focal point which is located in or near position 6. At position 17 a water cushion or any other kind of exit window for the acoustical energy is located.

FIG. 2 is a simplified depiction of a pressure pulse/shock wave generator, such as a shock wave head, with plane wave characteristics. Numeral 1 indicates the position of a pressure pulse generator according to the present invention, which generates a pressure pulse which is leaving the housing at the position 17, which may be a water cushion or any other kind of exit window. Somewhat even, also referred to herein as “disturbed”, wave characteristics can be generated, in case a paraboloid is used as a reflecting element, with a point source (e.g. electrode) that is located in the focal point of the paraboloid. The waves will be transmitted into the patient's body via a coupling media such as, e.g., ultrasound gel or oil and their amplitudes will be attenuated with increasing distance from the exit window 17.

FIG. 3 is a simplified depiction of a pressure pulse shock wave generator (shock wave head) with divergent wave characteristics. The divergent wave fronts may be leaving the exit window 17 at point 11 where the amplitude of the wave front is very high. This point 17 could be regarded as the source point for the pressure pulses. In FIG. 1c the pressure pulse source may be a point source, that is, the pressure pulse may be generated by an electrical discharge of an electrode under water between electrode tips. However, the pressure pulse may also be generated, for example, by an explosion, referred to as a ballistic pressure pulse. The divergent characteristics of the wave front may be a consequence of the mechanical setup.

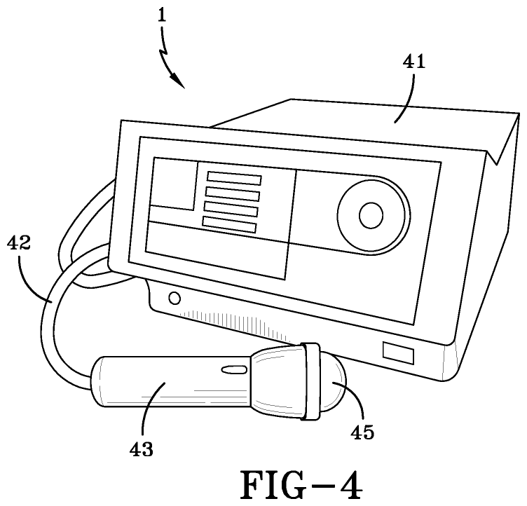

With reference to FIG. 4, an exemplary acoustic shock wave apparatus 1 is illustrated. The shock wave apparatus 1 has a generator 41 connected by a flexible hose with fluid conduits extending from the shock wave generator 41 to an applicator 43 which transmits the acoustic waves when coupled to the skin by using a fluid or acoustic gel. The applicator 43 as illustrated has a body that enables a technician to hold the applicator 43 and as illustrated this applicator is an electrohydraulic that is filled with fluid to facilitate the transmission of the shock waves. The fluid expands a flexible membrane in such a fashion that the membrane extends outwardly in a balloon shape fashion as illustrated in FIG. 4. As shown, this type of applicator 43 has a hydraulic spark generator using either focused or unfocused shock waves, preferably in a low energy level, less than the range of 0.01 mJ/mm² to 0.3 mJ/mm². The flexible hose 42 is connected to a fluid supply that fills the applicator 43 and expands the flexible membrane when filled. Alternatively, a ballistic, piezoelectric or spherical acoustic shock wave device can be used to generate the desired waves.

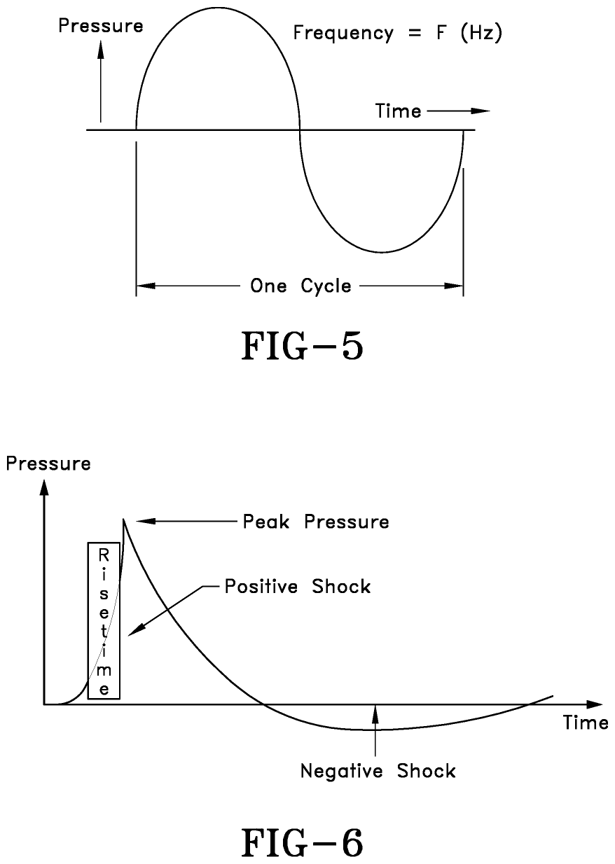

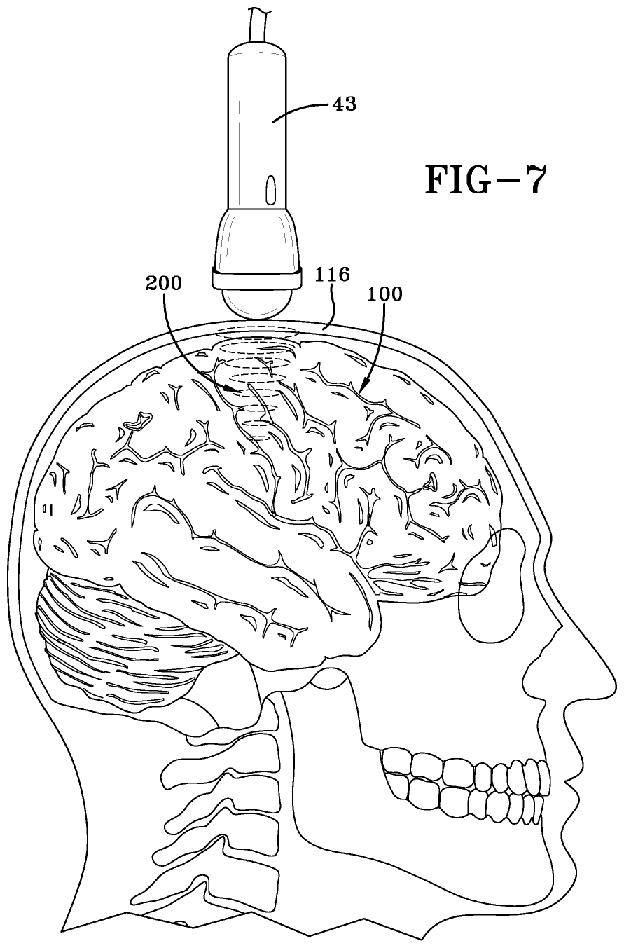

The ultrasonic wave pattern shown in FIG. 5 is contrasted to an asymmetric acoustic wave pattern which is illustrated in FIG. 6. As shown, ultrasound waves are symmetrical having the positive rise time equal to the negative in a sinusoidal wave form. These ultrasound waves generate heat in the tissue and are accordingly believed not suitable for use on organs or brain tissue.

FIG. 7 is a depiction of an acoustic shock wave treatment to a region of the brain 100 to reduce swelling or inflammation. The acoustic shock waves 200 are transmitted through the skin and skull 116 as shown.

This apparatus, in certain embodiments, may be adjusted/modified/or the complete shock wave head or part of it may be exchanged so that the desired and/or optimal acoustic profile such as one having wave fronts with focused, planar, nearly plane, convergent or divergent characteristics can be chosen.

This apparatus may, in certain embodiments, be adjusted/modified/or the complete shock wave head or part of it may be exchanged so that the desired and/or optimal acoustic profile such as one having wave fronts with focused, planar, nearly plane, convergent or divergent characteristics can be chosen.

A change of the wave front characteristics may, for example, be achieved by changing the distance of the exit acoustic window relative to the reflector, by changing the reflector geometry, by introducing certain lenses or by removing elements such as lenses that modify the waves produced by a pressure pulse/shock wave generating element. Exemplary pressure pulse/shock wave sources that can, for example, be exchanged for each other to allow an apparatus to generate waves having different wave front characteristics are described in detail below.

In certain embodiments, the change of the distance of the exit acoustic window can be accomplished by a sliding movement. However, in other embodiments of the present invention, in particular, if mechanical complex arrangements, the movement can be an exchange of mechanical elements.

In one embodiment, mechanical elements that are exchanged to achieve a change in wave front characteristics include the primary pressure pulse generating element, the focusing element, the reflecting element, the housing and the membrane. In another embodiment, the mechanical elements further include a closed fluid volume within the housing in which the pressure pulse is formed and transmitted through the exit window.

In one embodiment, the apparatus of the present invention is used in combination therapy. Here, the characteristics of waves emitted by the apparatus are switched from, for example, focused to divergent or from divergent with lower energy density to divergent with higher energy density. Thus, effects of a pressure pulse treatment can be optimized by using waves having different characteristics and/or energy densities, respectively.

While the above described universal toolbox of the present invention provides versatility, the person skilled in the art will appreciate that apparatuses that only produce waves having, for example, nearly plane characteristics, are less mechanically demanding and fulfill the requirements of many users.

As the person skilled in the art will also appreciate that embodiments shown in the drawings are independent of the generation principle and thus are valid for not only electro-hydraulic shock wave generation but also for, but not limited to, PP/SW generation based on electromagnetic, piezoceramic and ballistic principles. The pressure pulse generators may, in certain embodiments, be equipped with a water cushion that houses water which defines the path of pressure pulse waves that is, through which those waves are transmitted. In a preferred embodiment, a patient is coupled via ultrasound gel or oil to the acoustic exit window (17), which can, for example, be an acoustic transparent membrane, a water cushion, a plastic plate or a metal plate.

These shock wave energy transmissions are effective in stimulating a cellular response and can be accomplished without creating the cavitation bubbles in the tissue of the target site when employed in other than site targeted high energy focused transmissions. This effectively insures the brain tissue does not have to experience the sensation of hemorrhaging so common in the higher energy focused wave forms having a focal point at or within the targeted treatment site. Bleeding internally causes an increase in fluid pressure which can lead to increased brain damage. This can be completely avoided in this treatment protocol.

The fact that some if not all of the dosage can be at a low energy the common problem of localized hemorrhaging is reduced making it more practical to administer multiple dosages of waves from various orientations inside the mouth to further optimize the treatment and cellular stimulation of the target site. Heretofore focused high energy multiple treatments induced pain and discomfort to the patient. The use of low energy focused or un-focused waves at the target site enables multiple sequential treatments.

The present method may need precise site location and can be used in combination with such known devices as ultrasound, cat-scan or x-ray imaging if needed. The physician's general understanding of the anatomy of the patient may be sufficient to locate the target area to be treated. This is particularly true when the device is visually within the surgeon's line of sight and this permits the lens or cover of the emitting shock wave source to impinge on the affected brain tissue directly through a transmission enhancing gel, water or fluid medium during the pressure pulse or shock wave treatment. The treated area can withstand a far greater number of shock waves based on the selected energy level being emitted. For example, at very low energy levels the stimulation exposure can be provided over prolonged periods as much as 20 minutes if so desired. At higher energy levels the treatment duration can be shortened to less than a minute, less than a second if so desired. The limiting factor in the selected treatment dosage is avoidance or minimization of surrounding cell hemorrhaging and other kinds of damage to the surrounding cells or tissue while still providing a stimulating stem cell activation or a cellular release or activation of proteins such as brain derived neurotropic factor (BDNF) or VEGF and other growth factors while simultaneously germicidally attacking the degenerative tissue or infectious bacteria at the target site.

Due to the wide range of beneficial treatments available it is believed preferable that the optimal use of one or more wave generators or sources should be selected on the basis of the specific application. A key advantage of the present inventive methodology is that it is complimentary to conventional medical procedures. In the case of any operative surgical procedure the surgical area of the patient can be bombarded with these energy waves to stimulate cellular release of healing agents and growth factors. This will dramatically reduce the healing process time. Most preferably such patients may be provided more than one such treatment with an intervening dwell time for cellular relaxation prior to secondary and tertiary post operative treatments.

The underlying principle of these pressure pulse or shock wave therapy methods is to enrich the treatment area directly and to stimulate the body's own natural healing capability. This is accomplished by deploying shock waves to stimulate strong cells in the surrounding tissue to activate a variety of responses. The acoustic shock waves transmit or trigger what appears to be a cellular communication throughout the entire anatomical structure, this activates a generalized cellular response at the treatment site, in particular, but more interestingly a systemic response in areas more removed from the wave form pattern. This is believed to be one of the reasons molecular stimulation can be conducted at threshold energies heretofore believed to be well below those commonly accepted as required. Accordingly, not only can the energy intensity be reduced in some cases, but also the number of applied shock wave impulses can be lowered from several thousand to as few as one or more pulses and still yield a beneficial stimulating response. The key is to provide at least a sufficient amount of energy to activate healing reactions.

The use of shock waves as described above appears to involve factors such as thermal heating, light emission, electromagnetic field exposure, chemical releases in the cells as well as a microbiological response within the cells.

The unfocused shock waves can be of a divergent wave pattern, planar or near planar pattern preferably convergent diffused or far-sighted wave pattern, of a low peak pressure amplitude and density. Typically, the energy density values range as low as 0.000001 mJ/mm² and having a high end energy density of below 1.0 mJ/mm², preferably 0.20 mJ/mm² or less. The peak pressure amplitude of the positive part of the cycle should be above 1.0 and its duration is below 1-3 microseconds.

The treatment depth can vary from the surface to the full depth of the treated organ. The treatment site can be defined by a much larger treatment area than the 0.10-3.0 cm² commonly produced by focused waves. The above methodology is particularly well suited for surface as well as sub-surface soft tissue organ treatments like the brain.

While the above listed indications cited above are not exhaustive nor intended to be limiting, it is exemplary of the wide range of beneficial uses of high energy focused or low energy and amplitude unfocused divergent, planar or nearly planar shock waves, convergent shock waves, diffused shock waves or a combination of shock wave types in the treatment of humans and other mammals that are exposed to a neurological trauma or disease affecting the nervous system or are at high risk to be so exposed as the result of a high potential genetic pre-disposition to such diseases.

It will be appreciated that the apparatuses and processes of the present invention can have a variety of embodiments, only a few of which are disclosed herein. It will be apparent to the artisan that other embodiments exist and do not depart from the spirit of the invention. Thus, the described embodiments are illustrative and should not be construed as restrictive.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. A method of treating a traumatic brain injury to reduce pressure and inflammation using pressure pulses or shock waves comprises the steps of:
placing an applicator head of an acoustic shock wave or pressure pulse generator or source on a head near a swollen region at the brain injury;
coupling the applicator head directly or indirectly to an exposed surface of the skin and head near the swollen region; and
activating the generator or source to emit pressure pulses or acoustic shock waves through the skin and head to brain tissue exhibiting high pressure and inflammation to reduce the pressure and inflammation.

2. The method of claim 1, wherein the emitted pressure pulses or acoustic shock waves are transmitted in a pattern passing through the head to the brain.

3. The method of claim 1, wherein the emitted pressure pulses or acoustic shock waves pattern impinges the brain prior through a boney structure of a cranium or skull.

4. The method of claim 1, wherein the pressure pulse being an acoustic pulse which includes several cycles of positive and negative pressure.

5. The method of claim 4, wherein the pressure pulse has an amplitude of the positive part of such a cycle should be above 0.1 MPa and the time duration of the pressure pulse is from below a microsecond to about a second.

6. The method of claim 5, wherein the rise times of the positive part of the first pressure cycle in the range of nanoseconds (ns) up to some milliseconds (ms).

7. The method of claim 6 wherein the acoustic shock waves being very fast pressure pulses having amplitudes above 0.1 MPa and rise times of the amplitude being below 1000 ns.

8. The method of claim 3, wherein the duration of the shock wave is typically below 1-3 microseconds (μs) for the positive part of a cycle and typically above some microseconds for the negative part of a cycle.

9. The method of claim 3, wherein subjecting the brain to convergent, divergent, planar or near planar acoustic shock waves or pressure pulses in the absence of a focal point impinging the neuronal cells stimulating a cellular response in the absence of creating cavitation bubbles evidenced by not experiencing the sensation of hemorrhaging caused by the emitted waves or pulses in neuronal cells wherein the neuronal cells are positioned within a path of the emitted shock waves or pressure pulses; and away from any localized geometric focal volume or point of the emitted shock waves wherein the emitted shock waves or pressure pulses either have no geometric focal volume or point or have a focal volume or point ahead of the neuronal cells or beyond the neuronal cells thereby passing the emitted waves or pulses through the neuronal cells while avoiding having any localized focal point within the neuronal cells of the brain.

10. The method of claim 1, wherein the emitted pressure pulses or shock waves are convergent, divergent, planar or near planar and the pressure pulse shock wave generator or source is based on electro-hydraulic, electromagnetic, piezoceramic or ballistic wave generation having an energy density value ranging as low as 0.00001 mJ/mm² to a high end of below 1.0 mJ/mm².

11. The method of claim 10, wherein subjecting the brain directly to the acoustic shock waves having a low energy density of less than 1.0 mJ/mm² per shock wave stimulates said neuronal cells or brain tissue wherein the neuronal cells or brain tissue is positioned directly within a path of the emitted pressure pulses or acoustic shock waves in the absence of any focal point or if a focal point exists, the neuronal cells or brain tissue being treated is positioned away from any focal point.

12. The method of claim 11, wherein the energy density is selected to avoid any cell damage to the neuronal cells or brain tissue.

13. The method of claim 1, wherein treating the brain to stimulate by accelerating or increasing neuronal cell growth or regeneration wherein the administering is applied to a patient who has a pathological condition of the brain exhibiting damage caused by injury or disease such as diabetes, brain damage associated with stroke, and for the treatment of neurological disorders related to neurodegeneration, including Parkinson's disease, Alzheimer's disease and amyotrophic lateral sclerosis, multiple sclerosis and disseminated sclerosis, and for the treatment of bipolar disorder, depression, and schizophrenia any one of which has caused an increased pressure and inflammation which is reduced by the treatment.

14. The method of treating the brain of claim 1 stimulates by accelerating and increasing neuronal cell neurological brain tissue growth or regeneration or repair in addition to reducing brain tissue swelling and pressure and inflammation and wherein the neuronal cell or neurological brain tissue is from a mammal which is a human or an animal.
